(19) United States
(12) Patent Application Publication
(10) Pub. No.: US 2022/0145281 A1
(43) Pub. Date: May 12, 2022

(54) NEW VACCINAL STRATEGY TO PREVENT OR TREAT RHUMATOID ARTHRITIS

(71) Applicants: INSERM (Institut National de la Santé et de la Recherche Médicale), Paris (FR); UNIVERSITÉ D'AIX MARSEILLE, Marseille cedex 7 (FR); ASSISTANCE PUBLIQUE HÔPITAUX DE MARSEILLE, Marseille (FR)

(72) Inventors: Jean ROUDIER, Marseille cedex 09 (FR); Isabelle AUGER, Marseille cedix 09 (FR); Nathalie BALANDRAUD, Marseille cedex 09 (FR)

(21) Appl. No.: 17/435,267

(22) PCT Filed: Mar. 6, 2020
(86) PCT No.: PCT/EP2020/056015

§ 371 Date: Aug. 31, 2021

(30) Foreign Application Priority Data
Mar. 7, 2019 (EP) 19305265.1

(51) Int. Cl. C12N 9/78; A61P 19/02; A61K 38/50

(52) U.S. Cl. CPC C12N 9/78; C12Y 305/03015; A61K 38/50; A61P 19/02

(57) ABSTRACT

The present invention relates to field of treatment of rheumatoid arthritis. The inventors propose that PAD4, one of the enzymes which convert arginine into citrulline, is a target antigen for T cells that help the production of ACPA. They recently demonstrated that PAD immunization triggers anti-citrullinated fibrinogen antibody production in normal mice. Here, they demonstrate that the risk (OR) to develop RA associated with each of 12 HLA-DRB1 genotype correlates with the likelihood for the two HLA-DR molecules encoded by each genotype to bind at least one random peptide from PAD4, but not from citrullinated or native fibrinogen. PBLs from patients with RA, PsA and controls proliferate to PAD4 and they identify, notably, a peptide from PAD4, p8 (SEQ ID NO: 6), that stimulates T cells from RA patients and a few patients with PsA. Proliferative responses to p8 are associated with RA, shared epitope positive HLA-DR alleles and antibodies to PAD4. Thus the present invention relates to a peptide derived from the PAD4 protein and its use in the treatment and prevention of rheumatoid arthritis.

### FIELD OF THE INVENTION

Thus the present invention relates to a peptide derived from the PAD4 protein and its use in the treatment and prevention of rheumatoid arthritis.

### BACKGROUND OF THE INVENTION

The influence of HLA-DRB1 genes on the development of rheumatoid arthritis has been known since 1976, with the observation that lymphocytes from RA patients do not stimulate each other in mixed lymphocyte cultures (1).

Since that time, HLA-DRB1 genes and their products have been characterized and their function, peptide presentation to CD4 T cells, has been identified. HLA-DRB1 polymorphism has been described and it has been shown that HLA-DRB1 alleles associated with RA share a similar motif in the third hypervariable region of their (31 chain. This motif called «shared epitope» is present in every HLA-DRB1 allele associated with RA (2). Both HLA-DRB1 alleles expressed in an individual influence his/her risk to develop RA. In 2013, the inventors performed an HLA-DRB1 genotype RA association study on 3000 subjects and published a double entry table indicating the Odds ratios, ranging from 0.2 to 30, to develop RA for 106 of the 136 most common HLA-DRB1 genotypes in Southern France (3).

Similarly, the definition of RA kept improving with regular revisions of diagnosis criteria. The 2010 ACR criteria include the presence of RA characteristic anti-citrullinated peptide antibodies (ACPA) (4). ACPA recognize citrullin residues on many different proteins (5-8). Citrullin is a modified form of arginin obtained after a post translational modification called deimination and carried by enzymes called Peptidyl Arginyl Deiminases (PADs) (9). ACPAs are present in roughly two thirds of patients with RA. They often precede RA. However, how Shared Epitope positive HLA-DRB1 genes contribute to the development of RA is still unknown.

A straightforward explanation, the “Shared Epitope binds citrullinated peptide” hypothesis (SECIT) suggests that HLA-DR restricted T cells might help antibody responses to the many different citrullinated proteins recognized by ACPAs. In this model, RA associated HLA-DRB1 alleles are supposed to bind citrullinated peptides and present them to helper T cells. This hypothesis is supported by very limited binding data on one vimentin peptide (10) and contradicted by extensive binding data on hundreds of fibrinogen, vimentin, collagen II and Epstein Barr virus peptides under native and citrullinated form (11-13). Besides, helper T cells specific for the numerous citrullinated target proteins of ACPA are elusive. In short, T cells that help the production of ACPA and their target antigen(s) are still to be identified.

### SUMMARY OF THE INVENTION

Here, the inventors propose that PAD4, one of the enzymes which convert arginine into citrulline, is a target antigen for T cells that help the production of ACPA. They recently demonstrated that PAD immunization triggers anti-citrullinated fibrinogen antibody production in normal mice. Here, they demonstrate that the risk (OR) to develop RA associated with each of 12 HLA-DRB1 genotype correlates with the likelihood for the two HLA-DR molecules encoded by each genotype to bind at least one random peptide from PAD4, but not from citrullinated or native fibrinogen. PBLs from patients with RA, PsA and controls proliferate to PAD4 and they identify, notably, a peptide from PAD4, p8 (SEQ ID NO: 6), that stimulates T cells from RA patients and a few patients with PsA. Proliferative responses to p8 are associated with RA, shared epitope positive HLA-DR alleles and antibodies to PAD4.

Thus the present invention relates to a peptide derived from the PAD4 protein and its use in the treatment and prevention of rheumatoid arthritis. Particularly, the invention is defined by its claims.

### DETAILED DESCRIPTION OF THE INVENTION

### Peptides of the Invention

A first aspect of the invention relates to a peptide derived from the PAD4 protein of SEQ ID NO°: 2.

As used herein, the term “PAD4” for “peptidyl arginine deiminase 4” has its general meaning in the art and denotes a Ca2+-dependent enzyme that converts arginine residues into citrulline. PAD4 is widely believed to play a causative role in RA disease onset and progression because RA-associated mutations in the PAD4 gene have been identified in a variety of populations (A. Suzuki et al., Nat. Genet., 2003, 34: 395-402; T. Iwamoto et al., Rheumatology, 2006, 45: 804-807; and Y. H. Lee et al., Rheumatol. Int., 2007, 27: 827-233) and RA patients produce autoantibodies that recognize citrulline-containing proteins. It has already been shown that PAD4 is a conformation-dependent autoantigen in some RA patients (Y. Takizawa et al., Scand. J. Rheumatol., 2005, 3: 212-215). In humans, the PAD4 protein contains 663 amino acid residues (GenBank Accession Number: NP 036519.1), and has a nucleic acid sequence defined in SEQ ID NO: 1 and an amino acid sequence defined in SEQ ID NO. 2.

Nucleic acids sequence of PAD4 (SEQ ID NO: 1):
agccagaggg acgagctagc ccgacgatgg cccaggggac attgatccgt gtgaccccag agcagcccac
ccatgccgtg tgtgtgctgg gcaccttgac tcagcttgac atctgcagct ctgcccctga ggactgcacg tccttcagca
tcaacgcctc cccaggggtg gtcgtggata ttgcccacag ccctccagcc aagaagaaat ccacaggttc ctccacatgg
cccctggacc ctggggtaga ggtgaccctg acgatgaaag cggccagtgg tagcacaggc gaccagaagg ttcagatttc
atactacgga cccaagactc caccagtcaa agctctactc tacctcaccg cggtggaaat ctccctgtgc gcagacatca
cccgcaccgg caaagtgaag ccaaccagag ctgtgaaaga tcagaggacc tggacctggg gcccttgtgg acagggtgcc
atcctgctgg tgaactgtga cagagacaat ctcgaatctt ctgccatgga ctgcgaggat gatgaagtgc ttgacagcga
agacctgcag gacatgtcgc tgatgaccct gagcacgaag acccccaagg acttcttcac aaaccataca ctggtgctcc
acgtggccag gtctgagatg gacaaagtga gggtgtttca ggccacacgg ggcaaactgt cctccaagtg cagcgtagtc
ttgggtccca agtggccctc tcactacctg atggtccccg gtggaaagca caacatggac ttctacgtgg aggccctcgc
tttcccggac accgacttcc cggggctcat taccctcacc atctccctgc tggacacgtc caacctggag ctccccgagg
ctgtggtgtt ccaagacagc gtggtcttcc gcgtggcgcc ctggatcatg acccccaaca cccagccccc gcaggaggtg
tacgcgtgca gtatttttga aaatgaggac ttcctgaagt cagtgactac tctggccatg aaagccaagt gcaagctgac
catctgccct gaggaggaga acatggatga ccagtggatg caggatgaaa tggagatcgg ctacatccaa gccccacaca
aaacgctgcc cgtggtcttc gactctccaa ggaacagagg cctgaaggag tttcccatca aacgagtgat gggtccagat
tttggctatg taactcgagg gccccaaaca gggggtatca gtggactgga ctcctttggg aacctggaag tgagcccccc
agtcacagtc aggggcaagg aatacccgct gggcaggatt ctcttcgggg acagctgtta tcccagcaat gacagccggc
agatgcacca ggccctgcag gacttcctca gtgcccagca ggtgcaggcc cctgtgaagc tctattctga ctggctgtcc
gtgggccacg tggacgagtt cctgagcttt gtgccagcac ccgacaggaa gggcttccgg ctgctcctgg ccagccccag
gtcctgctac aaactgttcc aggagcagca gaatgagggc cacggggagg ccctgctgtt cgaagggatc aagaaaaaaa
aacagcagaa aataaagaac attctgtcaa acaagacatt gagagaacat aattcatttg tggagagatg catcgactgg
aaccgcgagc tgctgaagcg ggagctgggc ctggccgaga gtgacatcat tgacatcccg cagctcttca agctcaaaga
gttctctaag gcggaagctt ttttccccaa catggtgaac atgctggtgc tagggaagca cctgggcatc cccaagccct
tcgggcccgt catcaacggc cgctgctgcc tggaggagaa ggtgtgttcc ctgctggagc cactgggcct ccagtgcacc
ttcatcaacg acttcttcac ctaccacatc aggcatgggg aggtgcactg cggcaccaac gtgcgcagaa agcccttctc
cttcaagtgg tggaacatgg tgccctgagc ccatcttccc tggcgtcctc tccctcctgg ccagatgtcg ctgggtcctc
tgcagtgtgg caagcaagag ctcttgtgaa tattgtggct ccctgggggc ggccagccct cccagcagtg gcttgctttc
ttctcctgtg atgtcccagt ttcccactct gaagatccca acatggtcct agcactgcac actcagttct gctctaagaa
gctgcaataa agttttttta agtcactttg tac
Amino acids sequence of PAD4 (SEQ ID NO: 2):
MAQGTLIRVT PEQPTHAVCV LGTLTQLDIC SSAPEDCTSF SINASPGVVV
DIAHSPPAKK KSTGSSTWPL DPGVEVTLTM KAASGSTGDQ KVQISYYGPK
TPPVKALLYL TAVEISLCAD ITRTGKVKPT RAVKDQRTWT WGPCGQGAIL
LVNCDRDNLE SSAMDCEDDE VLDSEDLQDM SLMTLSTKTP KDFFTNHTLV
LHVARSEMDK VRVFQATRGK LSSKCSVVLG PKWPSHYLMV PGGKHNMDFY
VEALAFPDTD FPGLITLTIS LLDTSNLELP EAVVFQDSVV FRVAPWIMTP
NTQPPQEVYA CSIFENEDFL KSVTTLAMKA KCKLTICPEE ENMDDQWMQD
EMEIGYIQAP HKTLPVVFDS PRNRGLKEFP IKRVMGPDFG YVTRGPQTGG
ISGLDSFGNL EVSPPVTVRG KEYPLGRILF GDSCYPSNDS RQMHQALQDF
LSAQQVQAPV KLYSDWLSVG HVDEFLSFVP APDRKGFRLL LASPRSCYKL
FQEQQNEGHG EALLFEGIKK KKQQKIKNIL SNKTLREHNS FVERCIDWNR
ELLKRELGLA ESDIIDIPQL FKLKEFSKAE AFFPNMVNML VLGKHLGIPK
PFGPVINGRC CLEEKVCSLL EPLGLQCTFI NDFFTYHIRH GEVHCGTNVR
RKPFSFKWWN MVP

In a particular embodiment, the peptide derived from the PAD4 protein is a peptide which bind to the HLA-DRB1*0101, HLA-DRB1*0401, HLA-DRB1*0404, HLA-DRB1*0402 or HLA-DRB1*0701 molecules. Particularly, the peptide derived from the PAD4 protein binds to the HLA-DRB1*0401 or HLA-DRB1*0404 molecules.

In another particular embodiment, the peptide derived from PAD4 protein is a peptide described in the table 1.

Thus, in particular embodiment, the peptide derived from PAD4 protein consists of the amino acids sequence selected from the group consisting of SEQ ID NO: 6; SEQ ID NO: 8 or SEQ ID Nos: 10 to 72.

In a particular embodiment, the peptide derived from the PAD4 protein is a peptide of 15 to 25 amino acids. In a particular embodiment, the peptide derived from the PAD4 protein is a peptide of 15, 16, 17, 18, 19, 20, 21, 21, 23, 24 or 25 amino acids.

In another particular embodiment, the peptide derived from PAD4 protein is a peptide which comprises or consists of the amino acids sequence from the amino acids 61 to 100 of the SEQ ID NO°: 2.

In another particular embodiment, the peptide derived from PAD4 protein is a peptide having an amino acids sequence comprising or consisting to the amino acids sequence from the amino acids 61 to 100 of the SEQ ID NO°: 2. In particular the peptide of the invention has 60%, 61%, 62%, 63%, 64%, 65%, 66%, 67%, 68%, 69%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of identity over the peptide derived from PAD4 protein positioning from the amino acid 61 to 100 of the SEQ ID NO°: 2 and is still able to be efficiently presented by antigen presenting cells.

In a particular embodiment, the invention relates to peptide derived from PAD4 protein having 60%, 61%, 62%, 63%, 64%, 65%, 66%, 67%, 68%, 69%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of identity with the amino acid sequences SEQ ID NO: 3 or 4.

In a particular embodiment, the peptide derived from PAD4 comprises or consists of the amino acids sequence SEQ ID NO: 3: or SEQ ID NO: 4.

SEQ ID NO: 3:
KSTGSSTWPL DPGVEVTLTM KAASGSTGDQ KVQISYYGPK
SEQ ID NO: 4:
KSTGSSTWPL DPGVEVTLTM KVASGSTGDQ KVQISYYGPK

In another particular embodiment, the peptide derived from PAD4 protein comprises or consists of the amino acids sequence SEQ ID NO: 5: DPGVEVTLTMK-Xaa12-ASGSTGDQ wherein Xaa12 is an alanine (Ala or A), or a valine (Val or V) or a function-conservative variant thereof.

In a particular embodiment, the peptide derived from PAD4 protein comprises or consists of the amino acids sequences DPGVEVTLTMKAASGSTGDQ (SEQ ID NO: 6, peptide p8) or DPGVEVTLTMKVASGSTGDQ (SEQ ID NO: 7, peptide p8 derived) or a function-conservative variant thereof.

In a particular embodiment, the peptide derived from PAD4 protein comprises or consists of the amino acids sequences VRVFQATRGKLSSKCSVVLG (SEQ ID NO: 8, peptide p22) or a function-conservative variant thereof.

In one embodiment, the peptides of the invention comprises at least 60% of identity over the peptides of SEQ ID NO: 6 (p8) SEQ ID NO: 7 (p8 derived) or 8 (p22) described in the invention, even more particularly at least 61%, 62%, 63%, 64%, 65%, 66%, 67%, 68%, 69%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% and is still able to be efficiently presented by antigen presenting cells.

As used herein, the term “function-conservative variants” refers to those in which a given amino acid residue in a protein or enzyme has been changed (inserted, deleted or substituted) without altering the overall conformation and function of the polypeptide. Such variants include protein having amino acid alterations such as deletions, insertions and/or substitutions. A “deletion” refers to the absence of one or more amino acids in the protein. An “insertion” refers to the addition of one or more of amino acids in the protein. A “substitution” refers to the replacement of one or more amino acids by another amino acid residue in the protein. Typically, a given amino acid is replaced by an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, particularly at least 75%, more particularly at least 85%, still particularly at least 90%, and even more particularly at least 95%, and which has the same or substantially similar properties or functions as the native or parent protein to which it is compared. Two amino acid sequences are “substantially homologous” or “substantially similar” when greater than 80%, particularly greater than 85%, particularly greater than 90% of the amino acids are identical, or greater than about 90%, particularly greater than 95%, are similar (functionally identical) over the whole length of the shorter sequence. Particularly, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

In a particular embodiment, the peptides of the invention may contain one or two more amino acids at their C and N-terminal parts.

According to the invention, the peptides of the invention can be obtained by synthesizing the peptides according to methods for peptide synthesis known in the art.

Typically, the invention encompasses peptides substantially identical to peptides of Table 1 or peptide of SEQ ID NO: 3, SEQ ID NO: 4, SEQ ID NO: 6 (p8), SEQ ID NO: 7 (p8 derived) or SEQ ID NO: 8 (p22) in which one or more residues have been conservatively substituted with a functionally similar residue and which displays the functional aspects of the peptides described above i.e. being still able to be efficiently processed by antigen presenting cells in substantially the same way as a peptide consisting of the given amino acid sequence.

Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid or another.

The term “conservative substitution” also includes the use of a chemically derivatized residue in place of a non-derivatized residue. “Chemical derivative” refers to a patient peptide having one or more residues chemically derivatized by reaction of a functional side group.

Examples of such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derived to form O-acyl or O-alkyl derivatives. The imidazole nitrogen of histidine may be derived to form N-im-benzylhistidine. Chemical derivatives also include peptides which contain one or more naturally-occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine.

In some embodiments, the peptide of the invention is fusioned with a heterologous polypeptide. In some embodiments, the heterologous polypeptide is a cell-penetrating peptide, a Transactivator of Transcription (TAT) cell penetrating sequence, a cell permeable peptide or a membranous penetrating sequence.

The term “cell-penetrating peptides” is well known in the art and refers to cell penetrating sequences or membranous penetrating sequences such as penetratin, TAT mitochondrial penetrating sequence and compounds described in Bechara and Sagan, 2013; Jones and Sayers, 2012; Khafagy el and Morishita, 2012; and Malhi and Murthy, 2012.

In a particular embodiment, the heterologous polypeptide is the Transactivator of Transcription (TAT) cell penetrating sequence (GRKKRRQRRRPQ; SEQ ID NO: 9) originally derived from the cell-penetrating HIV tat peptide.

### Nucleic Acids, Vectors, Recombinant Host Cells and Uses Thereof

Another object of the invention relates to a nucleic acid sequence encoding a peptide derived from PAD4 protein according to the invention.

In a particular invention, the invention relates to a nucleic acid sequence encoding a peptide of SEQ ID NO: 6 (p8), SEQ ID NO: 7 (p8 derived) or SEQ ID NO: 8 (p22) or a function-conservative variant thereof according to the invention.

Another object of the invention relates to an expression vector comprising a nucleic acid sequence encoding a peptide derived from PAD4 protein according to the invention.

In still another object, the invention relates to an expression vector comprising a nucleic acid sequence encoding a peptide of SEQ ID NO: 6 (p8), SEQ ID NO: 7 (p8 derived) or SEQ ID NO: 8 (p22) or a function-conservative variant thereof according to the invention.

According to the invention, expression vectors suitable for use in the invention may comprise at least one expression control element operationally linked to the nucleic acid sequence. The expression control elements are inserted in the vector to control and regulate the expression of the nucleic acid sequence. Examples of expression control elements include, but are not limited to, lac system, operator and promoter regions of phage lambda, yeast promoters and promoters derived from polyoma, adenovirus, retrovirus, lentivirus or SV40. Additional preferred or required operational elements include, but are not limited to, leader sequence, termination codons, polyadenylation signals and any other sequences necessary or preferred for the appropriate transcription and subsequent translation of the nucleic acid sequence in the host system. It will be understood by one skilled in the art that the correct combination of required or preferred expression control elements will depend on the host system chosen. It will further be understood that the expression vector should contain additional elements necessary for the transfer and subsequent replication of the expression vector containing the nucleic acid sequence in the host system. Examples of such elements include, but are not limited to, origins of replication and selectable markers. It will further be understood by one skilled in the art that such vectors are easily constructed using conventional methods or commercially available.

Another object of the invention is a host cell comprising an expression vector as described here above.

According to the invention, examples of host cells that may be used are eukaryote cells, such as animal, plant, insect and yeast cells and prokaryotes cells, such as E. coli. The means by which the vector carrying the gene may be introduced into the cells include, but are not limited to, microinjection, electroporation, transduction, or transfection using DEAE-dextran, lipofection, calcium phosphate or other procedures known to one skilled in the art.

In a preferred embodiment, eukaryotic expression vectors that function in eukaryotic cells are used. Examples of such vectors include, but are not limited to, viral vectors such as retrovirus, adenovirus, adeno-associated virus, herpes virus, vaccinia virus, poxvirus, poliovirus; lentivirus, bacterial expression vectors, plasmids, such as pcDNA3 or the baculovirus transfer vectors. Preferred eukaryotic cell lines include, but are not limited to, COS cells, CHO cells, HeLa cells, NIH/3T3 cells, 293 cells (ATCC #CRL1573), T2 cells, dendritic cells, or monocytes.

### Therapeutic Use

A second object of the invention relates to a peptide derived from PAD4 protein, a nucleic acid sequence, a vector or a host cell as described here above for use as a medicament.

In a particular embodiment, the invention relates to a peptide of SEQ ID NO: 6 (p8), SEQ ID NO: 7 (p8 derive) or SEQ ID NO: 8 (p22) or a function-conservative variant thereof according to the invention for use as a medicament.

In another particular embodiment, the invention relates to a peptide derived from the PAD4 protein which binds to the HLA-DRB1*0101, HLA-DRB1*0401, HLA-DRB1*0404, HLA-DRB1*0402 or HLA-DRB1*0701 molecules for use as a medicament.

In a particular embodiment, the invention relates to a peptide, a nucleic acid sequence, a vector or a host cell as described here above for use as a vaccine.

In one embodiment a peptide derived from PAD4 protein, a nucleic acid sequence, a vector or a host cell as described here above may be used in the treatment of rheumatoid arthritis or psoriatic arthritis in a subject in need thereof.

Thus, in another embodiment, the invention relates to a peptide derived from PAD4 protein, a nucleic acid sequence, a vector or a host cell as described here above for use in the treatment of rheumatoid arthritis or psoriatic arthritis in a subject in need thereof.

In a particular embodiment, the invention relates to a peptide of SEQ ID NO: 6 (p8), SEQ ID NO: 7 (p8 derive) or SEQ ID NO: 8 (p22) or a function-conservative variant thereof according to the invention for use in the treatment of rheumatoid arthritis or psoriatic arthritis in a subject in need thereof.

As used herein, the term “subject” denotes a mammal, such as a rodent, a feline, a canine, and a primate. Particularly a subject according to the invention is a human.

Particularly, the subject in need thereof is a subject with HLA-DRB1*0101, HLA-DRB1*0401, HLA-DRB1*0404, HLA-DRB1*0402, HLA-DRB1*0701. Particularly, the subject has an HLA-DRB1*0401, HLA-DRB1*0404.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

A “therapeutically effective amount” as used herein is intended for a minimal amount of active agent which is necessary to impart therapeutic benefit to a patient. For example, a “therapeutically effective amount of the active agent” to a patient is an amount of the active agent that induces, ameliorates or causes an improvement in the pathological symptoms, disease progression, or physical conditions associated with the disease affecting the patient.

In a particular embodiment, the invention relates a method for treating or preventing rheumatoid arthritis or psoriatic arthritis by administrating to a subject in need thereof a peptide, a nucleic acid sequence, a vector or an host cell as described here above.

### Vaccine Composition and Uses Thereof

PAD4 is responsible of the citrullination of peptides which at the end of the process result in the production by the lymphocyte B of auto-antibodies against citrullinated peptides and thus to the disease.

Thanks to the results obtained by the inventors (see Results part), the inventor proposed here to use the peptides of the invention to inactive or decrease the activity of lymphocyte T which help lymphocytes B to produce auto-antibodies and thus to prevent and/or treat the disease by diminish or abolish the production of these auto-antibodies. The inventors show that these peptides (p8 and p22 notably), are presented by specific HLA-DR which for some, play a important role in the apparition and/or progression of the diseases. The peptides of the invention could thus be used in vaccine composition to tolerize a subject in need thereof.

Thus, a third aspect of the invention relates to a vaccine composition comprising a peptide derived from PAD4 protein, a nucleic acid sequence, a vector or an host cell as described here above.

In a particular embodiment, the vaccine composition comprises a peptide of SEQ ID NO: 6 (p8), SEQ ID NO: 7 (p8 derive) or SEQ ID NO: 8 (p22) or a function-conservative variant thereof according to the invention.

In one embodiment, the vaccine composition is useful to prevent or treat rheumatoid arthritis or psoriatic arthritis in a subject in need thereof.

In a particular embodiment, the vaccine composition may be useful to tolerizate the subject in need thereof.

In one embodiment, the vaccine composition is useful to diminish or abolish the production of auto-antibodies against citrullinated peptides.

As used herein, the term “tolerize” or “tolerization” denotes the fact of inducing tolerance by the stimulation of the formation of specific suppressor T lymphocytes. Thanks to this process and using peptides of the invention, a tolerance against these peptides will be induced and no or less antibodies against anti-citrullinated peptide will be generated. Indeed, PAD specific tolerization, at the opposite of PAD vaccination, does not intend to enhance anti PAD immune responses, but to diminish them or make them totally disappear. This can happen through the depletion, inactivation or death of PAD autoreactive T cells, or through the expansion of regulatory T cells.

The prophylactic administration of the vaccine composition of the invention should serve to prevent or attenuate disease like rheumatoid arthritis or psoriatic arthritis in a mammal. In a particular embodiment, mammals, particularly human, at high risk for rheumatoid arthritis or psoriatic arthritis are prophylactically treated with the vaccine composition of the invention. Examples of such mammals include, but are not limited to, humans with a family history of rheumatoid arthritis or psoriatic arthritis.

Thus, in one embodiment, the vaccine composition may be used as a therapeutic or prophylactic vaccine.

In one embodiment of the invention, the peptides of the invention may be conjugated with lipoprotein or administered in liposomal form or with adjuvant.

As used herein, the term “adjuvant” as used herein refers to a compound or a mixture that may be non-immunogenic when administered in the host alone, but that augments the host's immune response to an antigen when administered conjointly with that antigen.

In one embodiment, said vaccine composition is a pharmaceutical composition.

In such embodiment, said vaccine composition, for human use, comprises at least one antigen peptide as described here above or at least one antibody as described here above, together with one or more pharmaceutically acceptable carriers and, optionally, other therapeutic ingredients. The carrier(s) must be “acceptable” in the sense of being compatible with the other ingredients of the composition and not deleterious to the recipient thereof. The vaccine compositions may conveniently be presented in unit dosage form and may be prepared by any method well-known in the pharmaceutical art.

Vaccine compositions suitable for intravenous, intradermal, intramuscular, subcutaneous, or intraperitoneal administration conveniently comprise sterile aqueous solutions of the active agent with solutions which are particularly isotonic with the blood of the recipient. Such compositions may be conveniently prepared by dissolving solid active ingredient in water containing physiologically compatible substances such as sodium chloride (e.g. 0.1-2.0M), glycine, and the like, and having a buffered pH compatible with physiological conditions to produce an aqueous solution, and rendering said solution sterile. These may be present in unit or multi-dose containers, for example, sealed ampoules or vials.

The vaccine compositions of the invention may incorporate a stabilizer. Illustrative stabilizers are polyethylene glycol, proteins, saccharides, amino acids, inorganic acids, and organic acids which may be used either on their own or as admixtures. These stabilizers are particularly incorporated in an amount of 0.11-10,000 parts by weight per part by weight of active agent. If two or more stabilizers are to be used, their total amount is particularly within the range specified above. These stabilizers are used in aqueous solutions at the appropriate concentration and pH. The specific osmotic pressure of such aqueous solutions is generally in the range of 0.1-3.0 osmoles, particularly in the range of 0.8-1.2. The pH of the aqueous solution is adjusted to be within the range of 5.0-9.0, particularly within the range of 6-8.

Additional pharmaceutical methods may be employed to control the duration of action. Controlled release preparations may be achieved through the use of polymer to complex or absorb the peptides of the invention. The controlled delivery may be exercised by selecting appropriate macromolecules (for example polyester, polyamirio acids, polyvinyl, pyrrolidone, ethylenevinylacetate, methylcellulose, carboxymethylcellulose, or protamine sulfate) and the concentration of macromolecules as well as the methods of incorporation in order to control release. Another possible method to control the duration of action by controlled-release preparations is to incorporate the antigen peptides of the invention into particles of a polymeric material such as polyesters, polyamino acids, hydrogels, poly(lactic acid) or ethylene vinylaceiate copolymers. Alternatively, instead of incorporating these agents into polymeric particles, it is possible to entrap these materials in microcapsules prepared, for example, by coacervation techniques or by interfacial polymerization, for example, hydroxy-methylcellulose or gelatin-microcapsules and poly(methylmethacylate) microcapsules, respectively, or in colloidal drug delivery systems, for example, liposomes, albumin microspheres, microemulsions, nanoparticles, and nanocapsules or in macroemulsions.

When oral preparations are desired, the compositions may be combined with typical carriers, such as lactose, sucrose, starch, talc magnesium stearate, crystalline cellulose, methyl cellulose, carboxymethyl cellulose, glycerin, sodium alginate or gum arabic among others.

Vaccination of a patient with the vaccine composition of the invention can be conducted by conventional methods, for example, in the presence of conventional adjuvants. Examples of conventional adjuvant include, but are not limited to, metal salts, oil in water emulsions, Toll like receptors agonists, saponins, lipid A, alkyl glucosaminide phosphate, Freund's adjuvant, keyhole limpet haemocyanin (KLH), mannan, BCG, alum, cytokines such as IL-1, IL-2, macrophage colony stimulating factor, and tumor necrosis factor; and other substances that act as immunostimulating agents such as muramyl peptides or bacterial cell wall components, toxins, toxoids and TLR ligands.

The vaccine composition can be administered by any route appropriate such as intravenous, intraperitoneal, intramuscular, subcutaneous, intra-dermic and the like. The immunising composition may be administered once or at periodic intervals until a significant immune response is obtained (for example the apparition, after vaccination, of lymphocytes T CD4 and CD8 specific of the vaccine peptides used).

If the patient to be immunized is already afflicted with a rheumatoid arthritis or a psoriatic arthritis, the vaccine composition of the invention can be administered in conjunction with other therapeutic treatments. Examples of other therapeutic treatments includes, but are not limited to, adoptive T cell immunotherapy, coadministration of cytokines or other therapeutic drugs for rheumatoid arthritis or psoriatic arthritis like methotrexate, anti-TNF alpha, CTLA4 IgG (Abatacept), anti-CD20, anti-IL6 or anti-IL6 Receptor.

The dose of peptides of the invention to be administered to a patient may be adjusted as appropriate depending on, for example, the disease to be treated, the age and the body weight of said patient. Ranges of antigen peptides of the invention that may be administered are about 0.001 to about 100 mg per patient, preferred doses are about 0.01 to about 10 mg per patient.

The vaccine composition of the invention may be evaluated first in animal models, initially rodents, and in nonhuman primates and finally in humans. The safety of the immunization procedures is determined by looking for the effect of immunization on the general health of the immunized animal (weight change, fever, appetite behavior etc.) and looking for pathological changes on autopsies. After initial testing in animals, cancer patients can be tested. Conventional methods would be used to evaluate the immune response of the patient to determine the efficiency of the immunising composition.

### T Lymphocytes and Uses Thereof

Another object of the invention relates to a T lymphocyte that recognizes specifically a peptide of the invention.

In one embodiment of the invention, said T lymphocyte is a T CD4, T CD8 lymphocyte or a T helper lymphocyte.

In another embodiment of the invention, said T lymphocyte is a T cell clone.

In another embodiment, said T lymphocyte is a genetically modified T lymphocyte that expresses a TCR that recognizes specifically the peptide of the invention.

Another object of the invention is a composition for adoptive therapy comprising said T lymphocytes as described here above that recognizes specifically a peptide of the invention.

Particularly, the T lymphocytes can be used to prevent and treat rheumatoid arthritis or psoriatic arthritis in a subject in need thereof.

In a particular embodiment, the T cells are contained in physiological saline, phosphate buffered saline (PBS), culture medium, or the like in order to their stable maintain. Administration may be achieved, for example, intravenously.

Examples of where T-lymphocytes can be isolated, include but are not limited to, peripheral blood cells lymphocytes (PBL), lymph nodes, or tumor infiltrating lymphocytes (TIL).

Such lymphocytes can be isolated from peripheral blood of the individual to be treated by methods known in the art and cultured in vitro. Lymphocytes are cultured in media such as RPMI or RPMI 1640 for 2-5 weeks, particularly for 2-3 weeks. Viability is assessed by trypan blue dye exclusion assay. The lymphocytes are exposed to the antigen peptide of the invention for all of the culture duration.

In a preferred embodiment the lymphocytes are exposed to the peptides of the invention at a concentration of about 1 to about 10 micrograms (μm)/ml for all the duration of lymphocyte culture. Cytokines may be added to the lymphocyte culture such as IL-2.

The peptides of the invention may be added to the culture in presence of antigen presenting cells such as dendritic cells or allogeneic irradiated cancer cell line cells.

Particularly, the peptides of the invention may be added to the culture in presence of immunosuppressive molecules to obtained regulatory T cells which will

After being sensitized to the peptide, the T-lymphocytes are administered to the patient in need of such treatment.

Examples of how these sensitized T-cells can be administered to the mammal include but are not limited to, intravenously, intraperitoneally or intralesionally. Parameters that may be assessed to determine the efficacy of these sensitized T-lymphocytes include, but are not limited to, production of immune cells in the patient being treated or tumor regression. Conventional methods are used to assess these parameters. Such treatment can be given in conjunction with cytokines or gene modified cells (Rosenberg, S. A. et al. (1992) Human Gene Therapy, 3: 75-90; Rosenberg, S. A. et al. (1992) Human Gene Therapy, 3: 57-73).

Another object of the invention is a method for producing T lymphocytes that recognize specifically the peptides of the invention, said method comprising the steps of:

(a) stimulating peripheral blood mononuclear cells (PBMCs) or tumor infiltrating lymphocytes (TIL) obtained from a patient with at least one peptide of the invention,

(b) enriching the population of T lymphocytes specific for the peptide used in (a),

(c) optionally cloning said population of T lymphocytes specific for the peptide used in (a).

Enrichment and/or cloning may be carried out by using an MHC/peptide multimer as described here above. Cloning may also be carried out by conventional methods.

Stimulation of PBMCs may be carried out with at least one peptide of the invention alone, or presented by an antigen presenting cell such as dendritic cells or allogeneic irradiated cancer cell line cells. Typically, cytokines such as IL-2 may also be added to the culture.

Another object of the invention is a composition for adoptive therapy that comprises lymphocytes that recognizes specifically a peptide of the invention for preventing or treating disease like rheumatoid arthritis or psoriatic arthritis in a patient in need thereof, wherein said T lymphocytes are to be re-administrated to the patient.

The invention will be further illustrated by the following figures and examples. However, these examples and figures should not be interpreted in any way as limiting the scope of the present invention.

### FIGURES and TABLES

FIG. 1: Proliferative response to human PAD4 in 41 patients with RA, 25 patients with psoriatic arthritis and 11 healthy controls. 41 patients with RA, 25 patients with Psoriatic arthritis and 11 normal controls were tested for proliferative response to PAD4, native and citrullinated fibrinogen. For each protein, BRDU incorporation was tested in 4 replicates. Positivity was defined by a ratio between the OD for a well with cells and protein and the OD for a well with cells but without protein higher than 2 (OD ratio higher than 2). Mean OD ratios are indicated in red.

FIG. 2: Proliferative response to peptides from PAD4 in 22 patients with RA, 16 patients with psoriatic arthritis and 11 healthy controls. 22 patients with RA, 16 patients with Psoriatic arthritis and 11 healthy controls were tested for proliferative response to PAD4 peptides. For each peptide, BRDU incorporation was tested in 4 replicates. Positivity was defined by a ratio between the OD for a well with cells and peptide and the OD for a well with cells but without peptide higher than 2 (OD ratio higher than 2). Mean OD ratios are indicated in red.

FIG. 3: Antibody responses to human PAD4 in 41 patients with RA, 25 patients with psoriatic arthritis and 11 normal controls. 41 patients with ACPA positive RA, 25 patients with Psoriatic arthritis and 11 healthy controls were tested for anti PAD4 IgM and IgG by ELISA. Each antibody assay was performed in duplicate. Positivity was defined by a ratio between the OD for a well with serum and PAD4 protein and the OD for a well with serum but without PAD4 protein higher than 2 (OD ratio higher than 2) for IgG and higher than 3 for IgM. Mean OD ratios are indicated in red.

FIG. 4: antibody and proliferative responses to PAD4 in 41 patients with RA, 25 patients with Psoriatic arthritis and 11 healthy controls. 41 patients with ACPA positive RA, 25 patients with Psoriatic arthritis and 11 healthy controls were tested for T cell proliferation and antibody response to PAD4 and classified into 4 groups according to the results. SE: Shared epitope.

TABLE 2
Likelihood to bind PALM or fibrinogen peptides
for each of 12 different HLA-DRB1 genotypes.

| | | Likelihood to bind at least | | |
|---|---|---|---|---|
| | | one peptide from | | |
| | Risk to | | | Citrul- |
| HLA-DRB1* | develop RA | | Native | linated |
| Genotypes | (Odd Ratio) | PAD4 | fibrinogen | fibrinogen |
| DRB1*07/07 | 0.4 | 0.090 | 0.270 | 0.340 |
| DRB1*01:01/07 | 0.9 | 0.076 | 0.324 | 0.382 |
| DRB1*04:04/07 | 1.5 | 0.134 | 0.350 | 0.382 |
| DRB1*01:01/01:01 | 1.6 | 0.061 | 0.373 | 0.422 |
| DRB1*04:01/07 | 2.4 | 0.178 | 0.253 | 0.281 |
| DRB1*01:01/04:04 | 2.7 | 0.120 | 0.398 | 0.422 |
| DRB1*04:01/04:01 | 4.1 | 0.258 | 0.234 | 0.216 |
| DRB1*04:02/07 | 4.2 | 0.090 | 0.306 | 0.365 |
| DRB1*01:01/04:01 | 5 | 0.165 | 0.307 | 0.327 |
| DRB1*04:04/04:02 | 5.1 | 0.134 | 0.382 | 0.406 |
| DRB1*04:04/04:04 | 10.3 | 0.176 | 0.422 | 0.422 |
| DRB1*04:01/04:04 | 13.4 | 0.218 | 0.335 | 0.327 |
| Pearson | rho | 0.594 | 0.303 | −0.023 |
| | p | 0.042 | 0.339 | 0.945 |

TABLE 3
Subjects tested for Ab and T cell proliferation to PAD4.

| | RA | PsA | Healthy |
|---|---|---|---|
| Number | 41 | 25 | 11 |
| Sex | 30F | 18F | 8F |
| Disease duration (years) | 5 +/− 7 | 7 +/− 7 | |
| ACPA+ (3N) | 41/41 (100%) | 0 | 0 |
| RF+ | 24/33 (72%) | 1/15 (7%) | 0 |
| SE+ | 26/41 (63%) | 6/25 (24%) | 4/11 (36%) |
| Anti PAD4 Ig | 21/41 (51%) | 4/25 (16%) | 0 |
| Anti PAD4 LT | 19/41 (46%) | 8/25 (32%) | 4/11 (36%) |
| Treatment | 5 none | 4 none | |
| | 14 MTX | 1 MTX | |
| | 20 biotherapies | 16 biotherapies | |

RA: rheumatoid arthritis, PsA: psoriatic arthritis, F: female. ACPA: autoantibodies to citrullinated proteins, RF: rheumatoid factor. PALM: peptidylarginine deiminase, SE: shared epitope. MTX: methotrexate

TABLE 4
Subjects tested for immune responses to PAD4 and PAD4 peptides.

| | RA | PsA | Healthy |
|---|---|---|---|
| Number | 22 | 16 | 11 |
| Sex | 19F | 12F | 8F |
| Disease duration (years) | 7 +/− 7 | 8 +/− 7 | |
| ACPA+ (3N) | 22/22 (100%) | 0 | 0 |
| RF+ | 14/16 (87%) | 1/8 (12%) | 0 |
| SE+ | 14/22 (63%) | 6/16 (37.5%) | 4/11 (36%) |
| Anti PAD4 Ig | 10/22 (45%) | 3/16 (19%) | 0 |
| Anti PAD4 LT | 11/22 (50%) | 5/16 (31%) | 4/11 (36%) |
| Anti Peptide 8 LT | 9/22 (41%) | 3/16 (19%) | 0 |
| Treatment | 3 none | 2 none | |
| | 5 MTX | 1 MTX | |
| | 11 biotherapies | 10 biotherapies | |

RA: rheumatoid arthritis, PsA: psoriatic arthritis, F: female. ACPA: autoantibodies to citrullinated proteins, RF: rheumatoid factor. PALM: peptidylarginyl deiminase, SE: shared epitope. MTX: methotrexate.

### EXAMPLE

Material & Methods

Synthetic peptides from human PAD4 and human fibrinogen. Peptides were synthesized using the solid-phase system and purified (>70%)(Neosystem, Strasbourg, France). We synthesized 65 20-mers, encompassing residues 1-663 of wild-type PAD4 (residues S55, A82, and A112, on locus NM 012387) and overlapping on 10 amino acids. Residues S55, A82, and A112, which can be polymorphic, were detected under their native, unmutated form on peptides 5-6, 8-9, and 11-12, respectively. We synthesized 167 15-mers (71 native peptides, 71 citrullinated peptides and 25 peptides containing neither arginin nor citrulline) from the A and B chains of human fibrinogen (locus NP 000499, locus NP 005132). Whenever we thought the position of the R residue on the peptide might influence interaction with the P4 pocket, we synthesized extra, overlapping peptides (11).

Purification of HLA-DRB1 molecules from lymphoblastoid cell lines. The HLA homozygous lymphoblastoid cell lines JESTHOM (HLA-DRB1*01:01), SAVC (HLA-DRB1*04:01), YAR (HLA-DRB1*04:02), PEYSSON (HLA-DRB1*04:04), MOU (HLA-DRB1*07:01) were cultured in RPMI 1640 with 10% fetal calf serum. 2×109 cells were lysed in 10 mM Tris pH8, 10 mM NaCl, 10 mM MgCl2, 1% Triton X100, 0.05 mg/ml Dnase and protease inhibitors. These homozygous cell lines were chosen because they express 3 shared epitope positive alleles, HLA-DRB1*01:01, *04:01, *04:04 and two shared epitope negative alleles, not associated with RA, DRB1*04:02 and DRB1*07:01. Total protein extracts were immunoprecipitated by anti-HLA-DR LB3.1 antibody covalently coupled on cyanogen bromide-activated sepharose 4B (Sigma Aldrich, St. Quentin-Fallavier, France). After washing, HLA-DR molecules were eluted in PBS pH2 with 0.5% n-octylglucoside, neutralized in 1M Tris and quantified (11).

HLA-DR Peptide-binding assay. ELISA plates were coated with 10 μg of PAD4 or fibrinogen peptide/well and blocked in 1% bovine serum albumin (BSA). One microgram of purified HLA-DR molecule was added to plates. After washing, bound HLA-DR was detected by biotinylated anti HLA-DR antibody B8122 (Immunotech, Marseille, France), followed by peroxidase-conjugated avidin. Peroxidase-conjugated anti-mouse IgG and peroxidase-conjugated avidin were supplied by Sigma Aldrich (St. Quentin-Fallavier, France). Optical density was read at 405 nm. The binding of each of the purified HLA-DR alleles was assayed on ELISA plates coated with peptides from PAD4 or Fibrinogen (each in duplicate wells) and, as controls, 2 wells coated with a classical positive binder, influenza hemagglutinin (HA) peptide (PKYVKQNTLKLAT). Positive binding was defined as an OD value equal to the OD for the HA peptide.

Calculation of the likelihood to bind at least one peptide from a given protein for a particular HLA-DRB1 genotype. Briefly, we considered that the likelihood for a particular HLA-DRB1 allele to bind a given (unknown) peptide from a given protein was the ratio between number of bound peptides and total number of peptides from this protein. This ratio can be evaluated by studying the binding of a set of peptides covering the protein. Then, the likelihood for a given allele not to bind a peptide from the same protein is 1 minus the likelihood to bind a peptide. The likelihood for the two HLA-DRB1 alleles encoded by a genotype not to bind any peptide from the same protein is the product of the two likelihoods: (likelihood not to bind allele 1) (likelihood not to bind allele 2). Finally, the likelihood for the products of a given HLA-DRB1 genotype to bind at least one peptide from a protein is: 1 minus ((likelihood not to bind allele 1)×(likelihood not to bind allele 2)) (Table 2).

Patients. We tested 41 patients with rheumatoid arthritis (RA), 25 patients with psoriatic arthritis (PsA) from the rheumatology unit at Sainte Marguerite Hospital in Marseille, France and 11 healthy controls from the staff of the laboratory and the rheumatology ward. RA patients fulfilled the 2010 ACR/EULAR criteria and had ACPA titers higher than 3 times the upper limit of normal (4). Psoriatic arthritis patients fulfilled the the CASPAR criteria (26). HLA-DRB1 typing was performed by PCR/sequence specific oligonucleotide analysis in every patient and control (3). Anti-cyclic citrullinated peptide IgG antibodies were detected by a second-generation ELISA (Immunoscan RA Mark 2, Eurodiagnostica, Malmö, Sweden). Rheumatoid factors were detected by ELISA using the Orgentec Kit (Mainz, Germany). Baseline characteristics of patients are presented in Table 3 and Table 4.

Proteins. Human PAD4 protein was produced in baculovirus expression system and purified (Proteogenix, Schiltigheim, France). Activity and autocitrullination status were tested before T cell proliferation assay. Human fibrinogen (Merck Millipore, Darmstadt, Germany) was incubated in 1 M Tris HCl (pH7.4), 100 mM CaCl2, 50 mM dithiothreitol buffer at a concentration of 1 mg/ml with rabbit PAD2 protein (Sigma Aldrich, St. Quentin-Fallavier, France). Citrullination was performed for 2 hours at 37° C. Non citrullinated fibrinogen was treated identically, except that water was added instead of PAD.

T cell proliferation assay. Mononuclear cells from patients were isolated from 20 ml of heparinized blood by centrifugation through Ficoll-Histopaque (Sigma Aldrich, St. Quentin-Fallavier, France). Cells were cultured at a density of 106 cells/ml in RPMI 1640 with 10% self-serum in the presence of 1 μg/ml of human PAD4 or human fibrinogen or PHA (phytohemagglutinin) or 5 μg/ml of PAD4 peptide. After 6 days of culture at 37° C., proliferative response to proteins was evaluated using the colorimetric bromodeoxyuridine kit (Roche Diagnostics, Meylan, France). Positive T cell responses were defined by optical densities (OD) higher than twice the ODs for cells cultured without protein or peptide.

Detection of anti-PAD4 antibodies. Plates were coated with 0.5 μg human PAD4 and blocked with 2% BSA (bovine serum albumin). Sera diluted to 1:100 were incubated on plates. After washing, peroxydase conjugated anti-human IgG or IgM was added. Optical density (OD) was read at 405 nm. Background OD was obtained by adding each serum to a well without protein. Positive sera were defined by OD values higher than twice background OD for IgG and three times background OD for IgM.

Statistics. Correlation between the HLA-DRB1 genotypic Odds Ratio to develop RA and the likelihood to bind at least one PAD4 or Fibrinogen peptide for a given genotype was evaluated by Pearson's correlation test. Comparisons between groups for T cell and antibody assays were performed using the Fisher's test. GraphPad Prism 5.02 (GraphPad Software) was used for all statistical analyses.

Results

Binding of PAD4 and Fibrinogen Peptides to 5 Different HLA-DR Molecules

Binding of 65 peptides from PAD4, 96 citrullinated or arginine free peptides from fibrinogen and their 96 non citrullinated counterparts to 5 purified HLA-DR molecules was tested (Table 1 and data not shown). Each purified HLA-DRB1 molecule bound 2 to 9 of 65 PAD4 peptides, 12 to 23 of 96 fibrinogen peptides, 11 to 23 of 96 citrullinated fibrinogen peptides.

Likelihood to Bind PAD4 Peptide for Each of 12 Different HLA-DRB1 Genotypes

The likelihood to bind at least one PAD4 peptide, one native Fibrinogen peptide and one citrullinated Fibrinogen peptide was calculated for 12 genotypes for which we had calculated the risk to develop RA (Table 2).

Correlation Between OR to Develop RA and Likelihood to Bind at Least One PAD4 or Fibrinogen Peptide for 12 HLA-DRB1 Genotypes

There is a strong correlation between the risk to develop RA carried by each HLA-DRB1 genotype and the likelihood for its two encoded HLA-DRB1 molecules to bind at least one PAD4 peptide (p=0.042 Pearson's) (data not shown). Conversely, there is no correlation between the risk to develop RA carried by each HLA-DRB1 genotype and the likelihood for its two encoded HLA-DRB1 molecules to bind at least one peptide from native or citrullinated Fibrinogen (data not shown).

T Cell Proliferative Response to Human PAD4 is Common in Patients with RA, Psoriatic Arthritis and Normal Controls

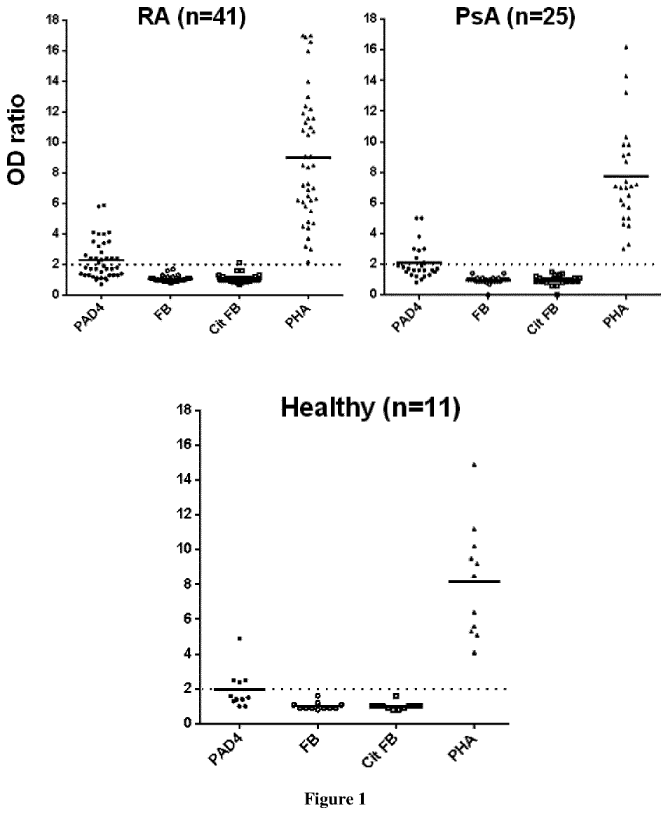

PBLs from 41 patients with ACPA positive RA, 25 patients with Psoriatic arthritis and 11 healthy controls were tested for T cell proliferative response to human PAD4, human native and citrullinated fibrinogen, by BRDU incorporation (Table 3, FIG. 1).

19/41 (46%) RA patients, 8/25 (32%) PsA patients, 4/11 (36%) controls had T cells proliferating to PAD4 (RA versus others, p=0.35 Fisher's test. N.S.).

One patient with RA (2.4%) and none of the patients with psoriatic arthritis or healthy controls had proliferative response to citrullinated fibrinogen. To confirm that the proliferative response observed in PBLs cultured with PAD4 was due to T cells, we ran flow cytometry analysis in an extra 10 RA patients and 7 controls. In this analysis, CD4 T cells showed increased expression of CD154, a T cell early activation marker and increased production of TNF alpha, in whole blood samples stimulated with PAD4 (data not shown).

T Cell Proliferative Responses to Peptides from PAD4 in 22 Patients with RA, 16 Patients with Psoriatic Arthritis and 11 Healthy Controls

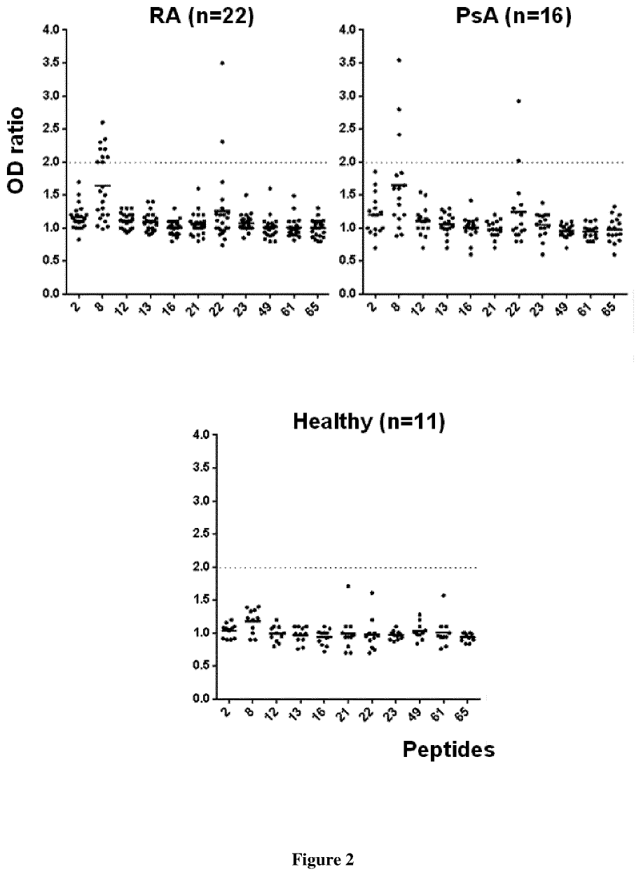

To identify which epitope(s) on PAD4 were recognized by T cells, we studied proliferative responses to 11 20 mers from PAD4 which we had found to be good binders of HLA-DRB1*04:01, *04:04, *01:01, *04:02 or *07:01, in 22 RA patients, 16 PsA patients and 11 healthy controls (Table 4, FIG. 2).

Proliferative response to peptide 22 (p22, sequence: VRVFQATRGKLSSKCSVVLG, SEQ ID NO: 8) a peptide found to bind HLA-DRB1*04:01 only), was observed in 2/22 (9%) RA patients, 2/16 (12.5%) patients with PsA and none of 11 controls (RA versus others, Fisher's test, N.S.).

Proliferative response to peptide 8 (p8, sequence: DPGVEVTLTMKAASGSTGDQ, SEQ ID NO: 6, a peptide that bound all five tested HLA-DRB1 alleles) was associated with RA: indeed 9/22 (41%) RA patients, 3/16 (19%) patients with PsA and 0/11 controls proliferated to p8 (RA versus others p=0.02, Fisher's test).

Proliferative response to peptide 8 was associated with the shared epitope: 75% (9/12) in responders against 40% (15 of 37) in non responders (responders against non responders p=0.05, Fisher's test).

Proliferative response to peptide 8 was associated with anti PAD4 antibodies (all subjects with proliferation to peptide 8 against all subjects with anti PAD4 antibodies p=0.05, Fisher's).

Antibody Responses to Human PAD4 in 41 Patients with RA, 25 with Psoriatic Arthritis and 11 Normal Controls

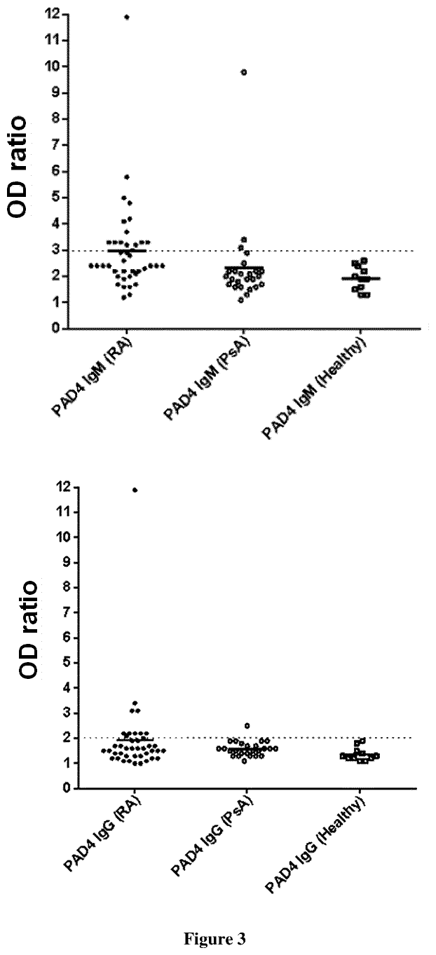

IgG anti PAD4 antibodies were detected in 11/41 (27%) patients with RA, 1/25 (4%) patients with PsA and 0/11 healthy controls (RA versus others, Fisher's test, p=0.004) (FIG. 3).

IgM antibodies to human PAD4 were detected in 14/41 (34%) patients with RA against 3 of 25 (12%) patients with psoriatic arthritis and none of 11 healthy controls (RA versus others, Fisher's test, p=0.01) (FIG. 3).

Positive anti PAD4 IgM responses in RA patients are most likely caused by recognition of IgM rheumatoid factors bound to IgG anti PAD4 antibodies by the peroxydase labelled anti IgM antibody used in the ELISA. Indeed, 11 of the 14 patients with RA who tested positive for anti PAD4 IgM also tested positive for IgM rheumatoid factors. Therefore the IgM anti PAD4 antibodies we detected in patients with RA most likely indicate the presence of IgG anti PAD4 antibodies in these patients. Thus, we pooled IgM and IgG anti PAD4 antibodies and called them “anti PAD4 antibodies”.

Anti PAD4 antibodies are present in 21/41 (51%) patients with RA, 4/25 (16%) patients with PsA and 0/11 healthy controls (RA versus others p=0.0002, Fisher's test).

13 of 14 Patients with Anti PAD4 Antibodies and T Cell Proliferation to PAD4 have RA

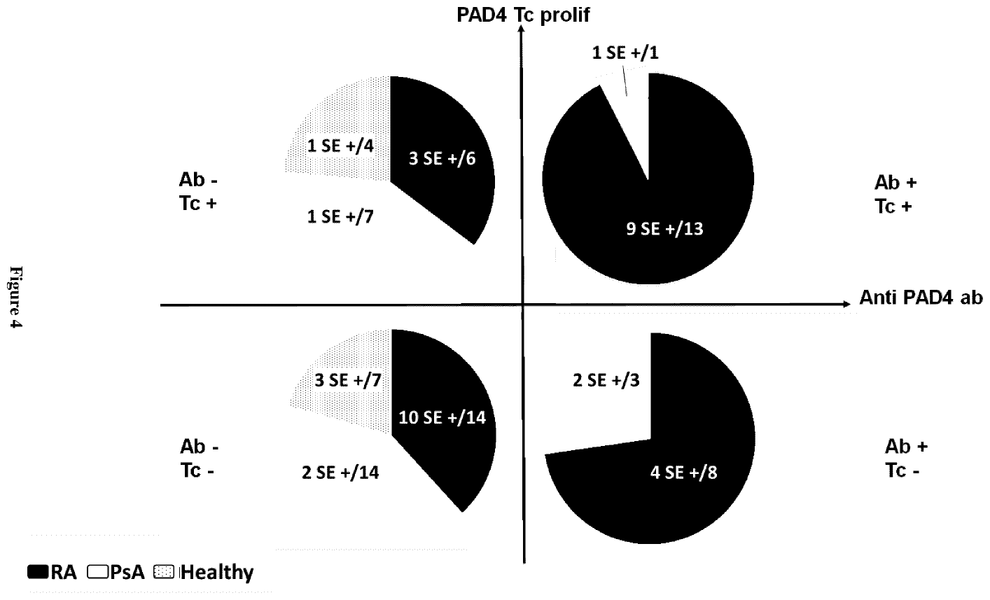

Among the 77 subjects that were tested, T cell proliferation and antibody response to PAD4 allowed us define 4 subgroups (FIG. 4):

35/77 subjects were negative for both antibody and proliferation to PAD4. They included 14 (34%) of 41 patients with RA, 14 (56%) of 25 patients with PsA and 7 (64%) of 11 healthy controls. RA patients were underrepresented in this group (RA versus others, p=0.04, Fisher's test.).

17/77 subjects were negative for antibodies and positive for T cell proliferation to PAD4. They included 6 (15%) of 41 patients with RA, 7 (28%) of 25 patients with PsA and 4 (36%) of 11 healthy controls (RA versus others, p=0.1, N. S.).

11/77 subjects were positive for antibodies and negative for T cell proliferation to PAD4. They included 8 (19.5%) of 41 patients with RA, 3 (12%) of 25 patients with PsA and none of 11 healthy controls (RA versus others, p=0.2, NS).

14/77 subjects were positive for both antibodies and T cell proliferation to PAD4. They included 13 (32%) of 41 patients with RA, 1 (4%) of 25 patients with PsA and none of 11 healthy controls (RA versus others, p=0.0009, Fisher's test) (FIG. 4).

In this “double positive” group, 71% (10 of 14) patients express the shared epitope against 41% in other groups (Fisher's, p=0.07).

Moreover, 25 had antibodies to PAD4, of whom 16 expressed SE positive HLA-DR alleles and 52 were negative for anti PAD4, of whom 20 expressed shared epitope positive HLA-DR alleles (Fisher's test, p=0.051) (FIG. 4).

31 subjects had proliferative response to PAD4, of whom 15 expressed SE positive HLADR alleles and 46 had no proliferative response to PAD4, of whom 21 expressed SE positive HLA-DR alleles (p=0.8, Fisher's test) (FIG. 4).

Thus, the HLA-DR SE is associated with anti PAD4 antibodies, but not with anti PAD4 proliferative response.

### Conclusion

The inventors have developed an alternative to the “Shared Epitope binds Citrullinated peptides” hypothesis. Indeed, the development of RA is preceded by the emergence of IgG antibodies to PAD4 (Peptidyl Arginyl Deiminase, the citrullinating enzyme) (17-21). This suggests the existence of helper T cells specific for PAD4 in patients with RA. Since PAD4 binds and citrullinates multiple proteins, any protein bound and being citrullinated by PAD4 may benefit from the help by a classical hapten carrier mechanism. Indeed, B cells specific for citrullinated residues on proteins bound to PAD4 could internalize and process the PAD4/citrullinated protein complex and present PAD4 peptides to helper T cells. This would lead to the production of IgG antibodies to multiple citrullinated proteins. To prove this point, the inventors have recently immunized normal non autoimmune mice with human and/or murine PADs and found that 20% of the mice developed anti citrullinated fibrinogen antibodies, in the absence of any T cell response to citrullinated fibrinogen (22).

Here, not knowing which peptide(s) from PAD4 or fibrinogen are critical to stimulate T cell responses, the inventors evaluated the likelihood for the 2 HLA-DR molecules encoded by each of 12 HLA-DRB1 genotypes to bind at least one of 65 peptides from the PAD4 protein and one of 96 native or 96 citrullinated peptides from human fibrinogen. To do so, the inventors studied the binding of overlapping peptides from PAD4 and native or citrullinated fibrinogen to 5 different HLA-DR alleles. The goal of this analysis was not to identify the actual peptide(s) from PAD4 or fibrinogen seen by helper T cells, but to quantify the likelihood for the two HLA-DR molecules encoded by a given genotype to bind an unknown relevant peptide from PAD4 or Fibrinogen. Regarding PAD4, a 663 amino acid protein, it can yield about 6500 relevant peptides (653 10mers, 652 11mers . . . 642 20 mers) and the inventors evaluated binding likelihoods using a sample of only 65 of them. Thus, this analysis can give us an indication of the ability for a pair of HLA-DR molecules to bind a peptide from a given protein, but not the certitude of having identified the relevant T cell epitope.

The inventors found a correlation between the likelihood that at least one the two HLA-DRB1 molecules encoded by each HLA-DRB1 genotype might bind at least one peptide from PAD4 and the OR to develop RA associated with this genotype. This was not observed with native or citrullinated peptides from fibrinogen.

The inventors then looked for T cell responses to PAD4 in the PBLs of patients with RA, psoriatic arthritis and healthy controls. They found that 19 of 41 patients with RA, 8 of 25 patients with PsA and 4 of 11 healthy controls had proliferative responses to PAD4. 13 of 41 RA patients had both proliferative response to PAD4 and anti PAD4 antibodies in their sera and this was different from all others (p=0.0009 Fisher's). This suggested that in these 13 patients, PAD4 specific T cells might provide help to PAD4 specific B cells. Thus, proliferative response to PAD4 is common, even in healthy subjects. However, when it is associated with anti PAD4 antibodies it is characteristic of ACPA positive RA. Among the 31 subjects (19 RA, 8 PsA and 4 controls) with proliferative responses to PAD4, SE HLA-DRB1 alleles were associated with anti PAD4 antibodies (Fisher's, p=0.03). This suggested that SE positive HLA-DRB1 alleles might allow PAD4 specific T cells to help anti PAD4 antibody production, maybe through binding of specific peptide(s).

To identity the peptides from PAD4 which are recognized by the T cells which proliferate to PAD4, the inventors performed proliferation studies with 11 peptides from PAD4 that were predicted to bind SE positive HLA-DRB1 alleles, in 22 patients with RA, 16 patients with PsA and 11 healthy controls. The inventors identified one particular peptide from PAD4, peptide 8 (p8), which was recognized by 9 of 22 RA patients, 3 of 16 patients with PsA and no healthy control. Proliferative response to p8 was associated with RA (p=0.02, Fisher's test), the HLA-DRB1 SE (p=0.05, Fisher's test) and antibodies to PAD4 (p=0.05, Fisher's test). The most straightforward explanation for these associations is that p8 may activate helper T cells. However, p8 is not a SE specific binder, it binds HLA-DRB1*04:01, 04:04, 01:01, 07:01, 04:02. Consistent with our binding data for p8, 12 of 13 patients with proliferative response to p8 express at least one of these 5 alleles. It is conceivable that presentation of p8 by SE positive HLA-DRB1 alleles might be more efficient than by shared epitope negative alleles, thus allowing activation of helper T cells. It is possible as well that p8 is better processed from PAD4 in subjects expressing SE positive than shared epitope negative HLA-DRB1 alleles, as already known of HLA-DRB1*04:01 (23).

Thus, the data of the inventors suggest a model for the development of ACPA in which T cells specific for PAD4 help, as expected, B cells specific for PAD4, leading to the production of IgG antibodies to PAD4, and at the same time, B cells specific for citrullinated antigens that have internalized and processed the PAD4/citrullinated antigen complex and present PAD4 peptide. These results place PAD4 at the center of the immunological conflict leading to RA. They remind us that the gene encoding PAD4 is associated with RA in Asians and in some European populations (24). Finally, if anti citrullinated protein immunity develops under the influence of helper T cells specific for peptide(s) from one protein, PAD4, as opposed to citrullinated peptide(s) from multiple proteins, it improves the prospects for prevention of RA by PAD4 vaccination in high risk individuals identified by their HLA-DRB1 genotypes.

In this respect, the identification of peptide 8 (and the others peptides of the invention) from PAD4 will be useful for peptide tolerization.

### REFERENCES

Throughout this application, various references describe the state of the art to which this invention pertains. The disclosures of these references are hereby incorporated by reference into the present disclosure.
1. Astorga G P, Williams R C Jr. Altered reactivity in mixed lymphocyte culture of lymphocytes from patients with rheumatoid arthritis. Arthritis Rheum. 1969; 12(6):547-54.
2. Gregersen P K, Silver J, Winchester R J. The shared epitope hypothesis: an approach to understanding the molecular genetics of susceptibility to rheumatoid arthritis. Arthritis Rheum. 1987; 30(11):1205-1213.
3. Balandraud N, et al. HLA-DRB1 genotypes and the risk of developing anti citrullinated protein antibody (ACPA) positive rheumatoid arthritis. PLoS One. 2013; 8(5):64108.
4. Aletaha D, et al. 2010 rheumatoid arthritis classification criteria: an American College of Rheumatology/European League Against Rheumatism collaborative initiative. Arthritis Rheum. 2010; 62(9): 2569-2581.
5. Simon M, et al. The cytokeratin filament aggregating protein filaggrin is the target of the so called “antikeratin antibodies,” autoantibodies specific for rheumatoid arthritis. J Clin Invest. 1993; 92(3): 1387-1393.
6. Girbal-Neuhauser E, et al. The epitopes targeted by the rheumatoid arthritis-associated antifilaggrin autoantibodies are posttranslationally generated on various sites of (pro)filaggrin by deimination of arginine residues. J Immunol. 1999; 162(1):585-594.
7. Vossenaar E, et al. Rheumatoid arthritis-specific anti-Sa antibodies target citrullinated vimentin. Arthritis Res Ther. 2004; 6(2):142-150.
8. Masson-Bessiere C, et al. The major synovial target of the rheumatoid arthritis-specific antifilaggrin autoantibodies are deiminated forms of the a and b chains of fibrin. J Immunol. 2001; 166(6). 4177-4184.
9. Schellekens G A, de Jong B A, van den Hoogen F H, van de Putte L B, van Venrooij W J. Citrulline is an essential constituent of antigenic determinants recognized by rheumatoid arthritis-specific autoantibodies. J Clin Invest. 1998; 101(1):273-281.
10. Hill J A, Southwood S, Sette A, Jevnikar A M, Bell D A, Cairns E. Cutting edge: The conversion of arginine to citrulline allows for a high-affinity peptide interaction with the rheumatoid arthritis associated HLA-DRB1*0401 MHC class II molecule. J Immunol. 2003; 171(2): 538-541.
11. Auger I, et al. Influence of HLA-DR genes on the production of rheumatoid arthritis-specific autoantibodies to citrullinated fibrinogen. Arthritis Rheum. 2005; 52 (11):3424-32.
12. Pratesi F, et al. Effect of rheumatoid arthritis (RA) susceptibility genes on the immune response to viral citrullinated peptides in RA. J Rheumatol. 2012; 39 (7):1490-1493.
13. Sidney J, Becart S, Zhou M, Duffy K, Lindvall M, Moore E C, et al. Citrullination only infrequently impacts peptide binding to HLA class II MHC. PLoS One. 2017; 12(5):e0177140.
14. Scally S W, et al. A molecular basis for the association of the HLA-DRB1 locus, citrullination, and rheumatoid arthritis. J Exp Med. 2013; 210(12):2569-2582.
15. Ting Y T, et al. The interplay between citrullination and HLA-DRB1 polymorphism in shaping binding hierarchies in rheumatoid arthritis. J Biol Chem. 2018; 293(9): 3236-3251.
16. James E A, et al. Citrulline-specific Th1 cells are increased in rheumatoid arthritis and their frequency is influenced by disease duration and therapy. Arthritis Rheumatol. 2014; 66(7): 1712-1722.
17. Kolfenbach J R, et al. Autoimmunity to peptidyl arginine deiminase type 4 precedes clinical onset of rheumatoid arthritis. Arthritis Rheum. 2010; 62(9):2633-2639.
18. Takizawa Y, Sawada T, Suzuki A, Yamada R, Inoue T, Yamamoto K. Peptidyl arginine deiminase 4 (PADI4) identified as a conformation-dependent autoantigen in rheumatoid arthritis. Scand J Rheumatol. 2005; 34(3):212-215.
19. Auger I, Balandraud N, Rak J, Lambert N, Martin M, Roudier J. New autoantigens in rheumatoid arthritis (RA): screening 8268 protein arrays with sera from patients with RA. Ann Rheum Dis. 2009; 68(4):591-594.
20. Charpin C, et al. New autoantibodies in early rheumatoid arthritis. Arthritis Res Ther. 2013; 15(4):R78
21. Auger I, Martin M, Balandraud N, Roudier J. RA specific autoantibodies to PAD4 inhibit citrullination of fibrinogen. Arthritis Rheum. 2010; 62 (1):126-131.
22. Arnoux F, et al. Peptidyl arginyl deiminase immunization induces anti citrullinated protein antibodies in mice with particular MHC types. Proc Natl Acad Sci USA. 2017; 114(47):E10169-E10177.
23. Roth S Willcox N, Rzepka R, Mayer M P, Melchers I. Major differences in antigen-processing correlate with a single Arg71<-->Lys substitution in HLA-DR molecules predisposing to rheumatoid arthritis and with their selective interactions with 70-kDa heat shock protein chaperones. J Immunol. 2002; 169(6):3015-20.
24. Suzuki A, et al. Functional haplotypes of PADI4, encoding citrullinating enzyme peptidylarginine deiminase 4, are associated with rheumatoid arthritis. Nat Genet. 2003; 34(4):395-402.
25. Darrah E, et al. Proteolysis by granzyme B enhances presentation of autoantigenic PAD4 epitopes in rheumatoid arthritis. J. Proteome Res. 2017; 16(1):355-365.
26. Chandran V. Spondyloarthritis: CASPAR criteria in early psoriatic arthritis. Nat Rev Rheumatol 2012; 8(9):503-504.

1. A peptide derived from the PAD4 protein of SEQ ID NO°: 2.

2. The peptide according to claim 1 wherein said peptide is described in table 1.

3. The peptide according to claim 1 wherein said peptide binds to a HLA-DRB1*0101, HLA-DRB1*0401, HLA-DRB1*0404, HLA-DRB1*0402 or HLA-DRB1*0701 molecule.

4. The peptide according to claim 1 wherein said peptide comprises or consists of the amino acid sequence of amino acids 61 to 100 of SEQ ID NO°: 2.

5. The peptide according to claim 4 wherein said peptide comprises or consists of the amino acid sequence SEQ ID NO: 3: or SEQ ID NO: 4.

6. The peptide according to claim 1 wherein said peptide comprises or consists of the amino acid sequence SEQ ID NO: 5: DPGVEVTLTMK-Xaa12-ASGSTGDQ wherein Xaa12 is an alanine (Ala or A), or a valine (Val or V) or a function-conservative variant thereof.

7. The peptide according to claim 6 wherein said peptide has the amino acid sequence SEQ ID NO: 6 or SEQ ID NO: 7 or a function-conservative variant thereof.

8. The peptide according to claim 1 wherein said peptide has the amino acid sequence SEQ ID NO: 8 or a function-conservative variant thereof.

9. A nucleic acid sequence encoding a peptide derived from PAD4 protein according to claim 1.

10. (canceled)

11. (canceled)

12. A method for treating or preventing rheumatoid arthritis or psoriatic arthritis comprising,
administrating to a subject in need thereof a therapeutically effective amount of a peptide according to claim 1 or a nucleic acid sequence encoding the peptide.

13. A vaccine composition comprising a peptide derived from PAD4 protein according to claim 1 or a nucleic acid sequence encoding the peptide.

14. The method of claim 12, wherein the peptide is administered in a vaccine composition.

15. A T lymphocyte that recognizes specifically a peptide according to claim 1.


SEQUENCE LISTING (72 sequences)

SEQ ID NO: 1 - DNA, 2263 bp, Homo sapiens
agccagagggacgagctagcccgacgatggcccaggggacattgatccgtgtgaccccagagcagcccacccatgccgtgtgtgtgctgggcaccttgactcagcttgacatctgcagctctgcccctgaggactgcacgtccttcagcatcaacgcctccccaggggtggtcgtggatattgcccacagccctccagccaagaagaaatccacaggttcctccacatggcccctggaccctggggtagaggtgaccctgacgatgaaagcggccagtggtagcacaggcgaccagaaggttcagatttcatactacggacccaagactccaccagtcaaagctctactctacctcaccgcggtggaaatctccctgtgcgcagacatcacccgcaccggcaaagtgaagccaaccagagctgtgaaagatcagaggacctggacctggggcccttgtggacagggtgccatcctgctggtgaactgtgacagagacaatctcgaatcttctgccatggactgcgaggatgatgaagtgcttgacagcgaagacctgcaggacatgtcgctgatgaccctgagcacgaagacccccaaggacttcttcacaaaccatacactggtgctccacgtggccaggtctgagatggacaaagtgagggtgtttcaggccacacggggcaaactgtcctccaagtgcagcgtagtcttgggtcccaagtggccctctcactacctgatggtccccggtggaaagcacaacatggacttctacgtggaggccctcgctttcccggacaccgacttcccggggctcattaccctcaccatctccctgctggacacgtccaacctggagctccccgaggctgtggtgttccaagacagcgtggtcttccgcgtggcgccctggatcatgacccccaacacccagcccccgcaggaggtgtacgcgtgcagtatttttgaaaatgaggacttcctgaagtcagtgactactctggccatgaaagccaagtgcaagctgaccatctgccctgaggaggagaacatggatgaccagtggatgcaggatgaaatggagatcggctacatccaagccccacacaaaacgctgcccgtggtcttcgactctccaaggaacagaggcctgaaggagtttcccatcaaacgagtgatgggtccagattttggctatgtaactcgagggccccaaacagggggtatcagtggactggactcctttgggaacctggaagtgagccccccagtcacagtcaggggcaaggaatacccgctgggcaggattctcttcggggacagctgttatcccagcaatgacagccggcagatgcaccaggccctgcaggacttcctcagtgcccagcaggtgcaggcccctgtgaagctctattctgactggctgtccgtgggccacgtggacgagttcctgagctttgtgccagcacccgacaggaagggcttccggctgctcctggccagccccaggtcctgctacaaactgttccaggagcagcagaatgagggccacggggaggccctgctgttcgaagggatcaagaaaaaaaaacagcagaaaataaagaacattctgtcaaacaagacattgagagaacataattcatttgtggagagatgcatcgactggaaccgcgagctgctgaagcgggagctgggcctggccgagagtgacatcattgacatcccgcagctcttcaagctcaaagagttctctaaggcggaagcttttttccccaacatggtgaacatgctggtgctagggaagcacctgggcatccccaagcccttcgggcccgtcatcaacggccgctgctgcctggaggagaaggtgtgttccctgctggagccactgggcctccagtgcaccttcatcaacgacttcttcacctaccacatcaggcatggggaggtgcactgcggcaccaacgtgcgcagaaagcccttctccttcaagtggtggaacatggtgccctgagcccatcttccctggcgtcctctccctcctggccagatgtcgctgggtcctctgcagtgtggcaagcaagagctcttgtgaatattgtggctccctgggggcggccagccctcccagcagtggcttgctttcttctcctgtgatgtcccagtttcccactctgaagatcccaacatggtcctagcactgcacactcagttctgctctaagaagctgcaataaagtttttttaagtcactttgtac

SEQ ID NO: 2 - protein, 663 aa, Homo sapiens
MAQGTLIRVTPEQPTHAVCVLGTLTQLDICSSAPEDCTSFSINASPGVVVDIAHSPPAKKKSTGSSTWPLDPGVEVTLTMKAASGSTGDQKVQISYYGPKTPPVKALLYLTAVEISLCADITRTGKVKPTRAVKDQRTWTWGPCGQGAILLVNCDRDNLESSAMDCEDDEVLDSEDLQDMSLMTLSTKTPKDFFTNHTLVLHVARSEMDKVRVFQATRGKLSSKCSVVLGPKWPSHYLMVPGGKHNMDFYVEALAFPDTDFPGLITLTISLLDTSNLELPEAVVFQDSVVFRVAPWIMTPNTQPPQEVYACSIFENEDFLKSVTTLAMKAKCKLTICPEEENMDDQWMQDEMEIGYIQAPHKTLPVVFDSPRNRGLKEFPIKRVMGPDFGYVTRGPQTGGISGLDSFGNLEVSPPVTVRGKEYPLGRILFGDSCYPSNDSRQMHQALQDFLSAQQVQAPVKLYSDWLSVGHVDEFLSFVPAPDRKGFRLLLASPRSCYKLFQEQQNEGHGEALLFEGIKKKKQQKIKNILSNKTLREHNSFVERCIDWNRELLKRELGLAESDIIDIPQLFKLKEFSKAEAFFPNMVNMLVLGKHLGIPKPFGPVINGRCCLEEKVCSLLEPLGLQCTFINDFFTYHIRHGEVHCGTNVRRKPFSFKWWNMVP

SEQ ID NO: 3 - protein, 40 aa, Artificial
  Synthetic amino acid 61 to 100-version 1
KSTGSSTWPLDPGVEVTLTMKAASGSTGDQKVQISYYGPK

SEQ ID NO: 4 - protein, 40 aa, Artificial
  Synthetic amino acid 61 to 100-version 2
KSTGSSTWPLDPGVEVTLTMKVASGSTGDQKVQISYYGPK

SEQ ID NO: 5 - protein, 20 aa, Artificial
  Synthetic Sequence p8 general
  MISC_FEATURE (12)..(12): A or V
DPGVEVTLTMKXASGSTGDQ

SEQ ID NO: 6 - protein, 20 aa, Artificial
  Synthetic peptide p8
DPGVEVTLTMKAASGSTGDQ

SEQ ID NO: 7 - protein, 20 aa, Artificial
  Synthetic Peptide p8 derived
DPGVEVTLTMKVASGSTGDQ

SEQ ID NO: 8 - protein, 20 aa, Artificial
  Synthetic Peptide 22
VRVFQATRGKLSSKCSVVLG

SEQ ID NO: 9 - protein, 12 aa, Artificial
  Synthetic peptide TAT
GRKKRRQRRRPQ

SEQ ID NO: 10 - protein, 20 aa, Artificial
  Synthetic peptide 1
MAQGTLIRVTPEQPTHAVCV

SEQ ID NO: 11 - protein, 20 aa, Artificial
  Synthetic peptide 2
PEQPTHAVCVLGTLTQLDIC

SEQ ID NO: 12 - protein, 20 aa, Artificial
  Synthetic peptide 3
LGTLTQLDICSSAPEDCTSF

SEQ ID NO: 13 - protein, 20 aa, Artificial
  Synthetic peptide 4
SSAPEDCTSFSINASPGVVV

SEQ ID NO: 14 - protein, 20 aa, Artificial
  Synthetic peptide 5
SINASPGVVVDIAHSPPAKK

SEQ ID NO: 15 - protein, 20 aa, Artificial
  Synthetic peptide 6
DIAHSPPAKKKSTGSSTWPL

SEQ ID NO: 16 - protein, 20 aa, Artificial
  Synthetic peptide 7
KSTGSSTWPLDPGVEVTLTM

SEQ ID NO: 17 - protein, 20 aa, Artificial
  Synthetic peptide 9
KAASGSTGDQKVQISYYGPK

SEQ ID NO: 18 - protein, 20 aa, Artificial
  Synthetic peptide 10
KVQISYYGPKTPPVKALLYL

SEQ ID NO: 19 - protein, 20 aa, Artificial
  Synthetic peptide 11
TPPVKALLYLTAVEISLCAD

SEQ ID NO: 20 - protein, 20 aa, Artificial
  Synthetic peptide 12
TAVEISLCADITRTGKVKPT

SEQ ID NO: 21 - protein, 20 aa, Artificial
  Synthetic peptide 13
ITRTGKVKPTRAVKDQRTWT

SEQ ID NO: 22 - protein, 20 aa, Artificial
  Synthetic peptide 14
RAVKDQRTWTWGPCGQGAIL

SEQ ID NO: 23 - protein, 20 aa, Artificial
  Synthetic peptide 15
WGPCGQGAILLVNCDRDNLE

SEQ ID NO: 24 - protein, 20 aa, Artificial
  Synthetic peptide 16
LVNCDRDNLESSAMDCEDDE

SEQ ID NO: 25 - protein, 20 aa, Artificial
  Synthetic peptide 17
SSAMDCEDDEVLDSEDLQDM

SEQ ID NO: 26 - protein, 20 aa, Artificial
  Synthetic peptide 18
VLDSEDLQDMSLMTLSTKTP

SEQ ID NO: 27 - protein, 20 aa, Artificial
  Synthetic peptide 19
SLMTLSTKTPKDFFTNHTLV

SEQ ID NO: 28 - protein, 20 aa, Artificial
  Synthetic peptide 20
KDFFTNHTLVLHVARSEMDK

SEQ ID NO: 29 - protein, 20 aa, Artificial
  Synthetic peptide 21
LHVARSEMDKVRVFQATRGK

SEQ ID NO: 30 - protein, 20 aa, Artificial
  Synthetic peptide 23
LSSKCSVVLGPKWPSHYLMV

SEQ ID NO: 31 - protein, 20 aa, Artificial
  Synthetic peptide 24
PKWPSHYLMVPGGKHNMDFY

SEQ ID NO: 32 - protein, 20 aa, Artificial
  Synthetic peptide 25
PGGKHNMDFYVEALAFPDTD

SEQ ID NO: 33 - protein, 20 aa, Artificial
  Synthetic peptide 26
VEALAFPDTDFPGLITLTIS

SEQ ID NO: 34 - protein, 20 aa, Artificial
  Synthetic peptide 27
FPGLITLTISLLDTSNLELP

SEQ ID NO: 35 - protein, 20 aa, Artificial
  Synthetic peptide 28
LLDTSNLELPEAVVFQDSVV

SEQ ID NO: 36 - protein, 20 aa, Artificial
  Synthetic peptide 29
EAVVFQDSVVFRVAPWIMTP

SEQ ID NO: 37 - protein, 20 aa, Artificial
  Synthetic peptide 30
FRVAPWIMTPNTQPPQEVYA

SEQ ID NO: 38 - protein, 20 aa, Artificial
  Synthetic peptide 31
NTQPPQEVYACSIFENEDFL

SEQ ID NO: 39 - protein, 20 aa, Artificial
  Synthetic peptide 32
CSIFENEDFLKSVTTLAMKA

SEQ ID NO: 40 - protein, 20 aa, Artificial
  Synthetic peptide 33
KSVTTLAMKAKCKLTICPEE

SEQ ID NO: 41 - protein, 20 aa, Artificial
  Synthetic peptide 34
KCKLTICPEEENMDDQWMQD

SEQ ID NO: 42 - protein, 20 aa, Artificial
  Synthetic peptide 35
ENMDDQWMQDEMEIGYIQAP

SEQ ID NO: 43 - protein, 20 aa, Artificial
  Synthetic peptide 36
EMEIGYIQAPHKTLPVVFDS

SEQ ID NO: 44 - protein, 20 aa, Artificial
  Synthetic peptide 37
HKTLPVVFDSPRNRGLKEFP

SEQ ID NO: 45 - protein, 20 aa, Artificial
  Synthetic peptide 38
PRNRGLKEFPIKRVMGPDFG

SEQ ID NO: 46 - protein, 20 aa, Artificial
  Synthetic peptide 39
IKRVMGPDFGYVTRGPQTGG

SEQ ID NO: 47 - protein, 20 aa, Artificial
  Synthetic peptide 40
YVTRGPQTGGISGLDSFGNL

SEQ ID NO: 48 - protein, 20 aa, Artificial
  Synthetic peptide 41
ISGLDSFGNLEVSPPVTVRG

SEQ ID NO: 49 - protein, 20 aa, Artificial
  Synthetic peptide 42
EVSPPVTVRGKEYPLGRILF

SEQ ID NO: 50 - protein, 20 aa, Artificial
  Synthetic peptide 43
KEYPLGRILFGDSCYPSNDS

SEQ ID NO: 51 - protein, 20 aa, Artificial
  Synthetic peptide 44
GDSCYPSNDSRQMHQALQDF

SEQ ID NO: 52 - protein, 20 aa, Artificial
  Synthetic peptide 45
RQMHQALQDFLSAQQVQAPV

SEQ ID NO: 53 - protein, 20 aa, Artificial
  Synthetic peptide 46
LSAQQVQAPVKLYSDWLSVG

SEQ ID NO: 54 - protein, 20 aa, Artificial
  Synthetic peptide 47
KLYSDWLSVGHVDEFLSFVP

SEQ ID NO: 55 - protein, 20 aa, Artificial
  Synthetic peptide 48
HVDEFLSFVPAPDRKGFRLL

SEQ ID NO: 56 - protein, 20 aa, Artificial
  Synthetic peptide 49
APDRKGFRLLLASPRSCYKL

SEQ ID NO: 57 - protein, 20 aa, Artificial
  Synthetic peptide 50
LASPRSCYKLFQEQQNEGHG

SEQ ID NO: 58 - protein, 20 aa, Artificial
  Synthetic peptide 51
FQEQQNEGHGEALLFEGIKK

SEQ ID NO: 59 - protein, 20 aa, Artificial
  Synthetic peptide 52
EALLFEGIKKKKQQKIKNIL

SEQ ID NO: 60 - protein, 20 aa, Artificial
  Synthetic peptide 53
KKQQKIKNILSNKTLREHNS

SEQ ID NO: 61 - protein, 20 aa, Artificial
  Synthetic peptide 54
SNKTLREHNSFVERCIDWNR

SEQ ID NO: 62 - protein, 20 aa, Artificial
  Synthetic peptide 55
FVERCIDWNRELLKRELGLA

SEQ ID NO: 63 - protein, 20 aa, Artificial
  Synthetic peptide 56
ELLKRELGLAESDIIDIPQL

SEQ ID NO: 64 - protein, 20 aa, Artificial
  Synthetic peptide 57
ESDIIDIPQLFKLKEFSKAE

SEQ ID NO: 65 - protein, 20 aa, Artificial
  Synthetic peptide 58
FKLKEFSKAEAFFPNMVNML

SEQ ID NO: 66 - protein, 20 aa, Artificial
  Synthetic peptide 59
AFFPNMVNMLVLGKHLGIPK

SEQ ID NO: 67 - protein, 20 aa, Artificial
  Synthetic peptide 60
VLGKHLGIPKPFGPVINGRC

SEQ ID NO: 68 - protein, 20 aa, Artificial
  Synthetic peptide 61
PFGPVINGRCCLEEKVCSLL

SEQ ID NO: 69 - protein, 20 aa, Artificial
  Synthetic peptide 62
CLEEKVCSLLEPLGLQCTFI

SEQ ID NO: 70 - protein, 20 aa, Artificial
  Synthetic peptide 63
EPLGLQCTFINDFFTYHIRH

SEQ ID NO: 71 - protein, 20 aa, Artificial
  Synthetic peptide 64
NDFFTYHIRHGEVHCGTNVR

SEQ ID NO: 72 - protein, 23 aa, Artificial
  Synthetic peptide 65
GEVHCGTNVRRKPFSFKWWNMVP